(19) United States
(12) Patent Application Publication
(10) Pub. No.: US 2025/0216375 A1
(43) Pub. Date: Jul. 3, 2025

(54) DETERMINISTIC ANALYSIS OF CONTAMINATION INFORMATION FLUID

(71) Applicant: Halliburton Energy Services, Inc., Houston, TX (US)

(72) Inventors: Christopher Michael JONES, Houston, TX (US); Bin DAI, Katy, TX (US); Rohin NAVEENA-CHANDRAN, Houston, TX (US); James MARTIN PRICE, Cypress, TX (US)

(73) Assignee: Halliburton Energy Services, Inc., Houston, TX (US)

(21) Appl. No.: 18/403,035

(22) Filed: Jan. 3, 2024

(51) Int. Cl. G01N 33/28; E21B 49/10; G01N 21/31

(52) U.S. Cl. CPC G01N 33/2823; E21B 49/10; G01N 21/31

(57) ABSTRACT

Systems and methods for determining a level of contamination of a formation fluid by a drilling fluid are disclosed. The method includes defining a first curve of a range of values for a plurality of properties of the formation fluid in a multidimensional space, wherein each dimension is associated with one of the plurality of properties, defining a first point of the values of the plurality of properties for the drilling fluid in the multidimensional space, accessing measurements of the plurality of properties for a plurality of samples of a fluid being pumped out of a wellbore, defining a plurality of second points of the respective measured properties in the multidimensional space, and defining a vector from the first point through the plurality of second points to the first curve. The method also includes determining a first distance from the first point along the vector to the first curve, determining a second distance from the most recent second point to the first curve, calculating a ratio of the second distance to the first distance, and providing the ratio as the level of contamination of the formation fluid by the drilling fluid.

### TECHNICAL FIELD

The present technology pertains to estimation of contamination of formation fluid by drilling fluid in pump-out from a well.

### BACKGROUND

During oil and gas exploration, many types of information may be collected and analyzed. The information may be used to determine the quantity and quality of hydrocarbons in a reservoir and to develop or modify strategies for hydrocarbon production. For instance, the information may be used for reservoir evaluation, flow assurance, reservoir stimulation, facility enhancement, production enhancement strategies, and reserve estimation. One technique for collecting relevant information involves obtaining and analyzing fluid samples from a reservoir of interest. There are a variety of different tools that may be used to obtain the fluid sample. The fluid sample may then be analyzed to determine fluid properties.

Current methods for analyzing water-based contamination in water-based formation fluids largely rely on statistical techniques and trend fitting. These methods involve collecting a series of measurements during the pump-out process and analyzing trends in these data to estimate the level of contamination. Trend fitting relies on several assumptions and are largely empirical and require a significant amount of user experience to be effective.

### BRIEF DESCRIPTION OF THE DRAWINGS

In order to describe the manner in which the features and advantages of this disclosure can be obtained, a more particular description is provided with reference to specific embodiments thereof which are illustrated in the appended drawings. Understanding that these drawings depict only exemplary embodiments of the disclosure and are not therefore to be considered to be limiting of its scope, the principles herein are described and explained with additional specificity and detail through the use of the accompanying drawings in which:

FIG. 1 is a schematic diagram of an example of a fluid sampling tool on a wireline, in accordance with various aspects of the subject technology.

FIG. 2 is a schematic diagram of an example of the fluid sampling tool on a drill string, in accordance with various aspects of the subject technology.

FIG. 3 is a schematic of an information handling system, in accordance with various aspects of the subject technology.

FIG. 4 is a schematic of a chipset that may be utilized by the information handling system, in accordance with various aspects of the subject technology.

FIG. 5 is a schematic of a schematic for an arrangement of resources on a computer network, in accordance with various aspects of the subject technology.

FIG. 6 is a schematic of the fluid sampling tool, in accordance with various aspects of the subject technology.

FIG. 7 depicts a hardware configuration of a dynamic subsurface optical measurement tool, in accordance with various aspects of the subject technology.

FIG. 8 is a workflow in accordance with certain embodiments, in accordance with various aspects of the subject technology.

FIG. 9 is a graph illustrating an optical spectrum of water, in accordance with various aspects of the subject technology.

FIG. 10 is a graph illustrating a deconvolved optical spectrum of water, in accordance with various aspects of the subject technology.

FIG. 11 is a graph illustrating a 2D example method of determining a level of contamination, in accordance with various aspects of the subject technology.

FIG. 12 is a flowchart illustrating an example method of determining a level of contamination, in accordance with various aspects of the subject technology.

### DETAILED DESCRIPTION

Obtaining clean samples of formation water is of paramount importance in the oil and gas industry. These samples provide critical information about the reservoir, including its salinity, pH, and the concentrations of various ions, which are essential for understanding the reservoir's geochemical conditions and predicting its behavior during production. Accurate formation water analysis can inform a range of operational decisions, from well completion design to production strategy, and can significantly impact the economic viability of a project. Furthermore, formation water composition plays a crucial role in flow assurance—the prediction, prevention, and remediation of issues that could impede fluid flow. For example, the presence of certain ions in formation water can lead to scaling or corrosion in the wellbore and production equipment, potentially causing costly damage and downtime. Therefore, obtaining clean samples of the formation water, i.e., free from drilling fluid contamination, is a significant economic consideration.

The current methods for analyzing contamination in water-based formation fluids largely rely on statistical techniques and trend fitting. These methods involve collecting a series of samples during the pump-out process and measuring one or more properties of the fluid, e.g., fluid color, density, viscosity, and/or pH, and analyzing the trends in this data to estimate the level of contamination. For example, one might observe how the density or pH of the fluid changes over time and fit a trend line to measurements. Trend fitting relies on three assumptions: 1) that the trend can be described by some function, 2) that the function remain valid throughout the entire process, which inherently requires that the processes of clean-up be unchanging, and 3) that the asymptote be representative of the pure formation fluid and not a steady-state contamination value. The trend is usually asymptotic with time or volume of the pump-out and the asymptote of the trend line is used to estimate the properties of the pure formation fluid and the level of contamination. Multivariate statistical techniques, such as principal component analysis, can also be used to analyze multiple properties simultaneously and identify the most significant trends in time or volume. These methods provide a more nuanced understanding of the contamination process and can help to improve the accuracy of contamination estimates. However, they are largely empirical and require a good amount of user experience to be effective.

A deterministic method for analyzing contamination in formation fluids offers several advantages over empirical, trend-fitting methods. Firstly, deterministic methods provide a clear, mechanistic understanding of the contamination process, which can lead to more accurate and reliable estimates of contamination levels. They allow for the direct calculation of contamination based on measured properties of the fluid, eliminating the need for trend fitting or statistical analysis. This can reduce the uncertainty associated with these methods and provide more precise estimates of formation fluid properties. Furthermore, deterministic methods can provide real-time feedback during the pumpout process, enabling immediate adjustments to minimize contamination. They also allow for the prediction of contamination levels under different conditions, which can be invaluable for planning and optimizing formation testing operations. Overall, a deterministic approach can provide a more robust and reliable framework for contamination estimation, leading to improved formation fluid sampling and analysis. The present embodiment proposes a method to overcome these challenges.

Various embodiments of the disclosure are discussed in detail below. While specific implementations are discussed, it should be understood that this is done for illustration purposes only. A person skilled in the relevant art will recognize that other components and configurations may be used without parting from the spirit and scope of the disclosure.

Additional features and advantages of the disclosure will be set forth in the description which follows, and in part will be obvious from the description, or can be learned by practice of the principles disclosed herein. The features and advantages of the disclosure can be realized and obtained by means of the instruments and combinations particularly pointed out in the appended claims. These and other features of the disclosure will become more fully apparent from the following description and appended claims or can be learned by the practice of the principles set forth herein.

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures, and components have not been described in detail so as not to obscure the related relevant feature being described. The drawings are not necessarily to scale and the proportions of certain parts may be exaggerated to better illustrate details and features. The description is not to be considered as limiting the scope of the embodiments described herein.

### Downhole Fluid Analysis

Disclosed herein are methods and systems for sampling reservoir water and, more particularly, disclosed are methods and systems for performing downhole fluid analysis. Even more particularly, certain methodologies in accordance with the present disclosure use a downhole spectroscopy device with a Fluid Sampler (commercial name ICS) to determine concentration of ions. An optical spectrum in a near infrared region corresponding to water (wavelengths around 1420 nm) may be split into two components using spectral deconvolution. Equations that may be used to carry out the spectral deconvolution include Gaussian, Lorenzian, or Voight Curve with Gaussian and Lorenzian addition. Once this spectrum has been split (deconvolved), there are unique characteristics of the Gaussian and Lorenzian curves that correspond to different concentrations of ions. These are one or more of a peak height, Full Width Half Max (FWHM), or the integrated area of a component. By using these components in combination with temperature, a separate equation (model) can be realized which may be used to calculate ion concentration. Downhole sampling of a reservoir fluid may be performed to carry out certain embodiments of the present disclosure. Generally speaking, downhole sampling refers to a type of downhole operation and which may be used for formation evaluation, asset decisions, and operational decisions. In general, a fluid sampling tool is utilized for analyzing the fluids from a formation and their composition. Water sampling is often not a priority or performed. Additionally, if water sampling is performed, the water sample is analyzed at a lab at surface. Methods and systems discussed herein allow water sampling to be performed downhole, to perform in-situ analysis of fluid and ionic properties of samples, and to distinguish between different water types.

As used herein, a “property” of a fluid refers to a chemical property, phase property, i.e., liquid or gas or solid phase in concentration or identification, or phase behavior. Examples of properties include: compositional component concentrations, e.g., methane, ethane, propane, butane, and pentane; organic liquid components, e.g., a hexane plus (C6+) fraction or hydrocarbon components therein, saturates fraction, aromatics fraction, resins fraction, asphaltenes fraction; total acid number; pH, eH (activity of electrons), water composition, including cations such as sodium, potassium, calcium, magnesium and trace cations, anions such as chloride, bromide, sulfide, sulfate, carbonate, bicarbonate, other dissolved solids; organic acids and/or their conjugates; and other inorganic components such as carbon dioxide, hydrogen sulfide, nitrogen or water. Physical properties include compressibility, density, thermal conductivity, heat capacity, viscosity; phase behavior including bubble point, gas to oil ratio, phase envelope for gas-liquid or solid-liquid, including asphaltenes or waxes; and compositional grading with depth. Properties also include the interpretation of similarity or differences between different set fluids such as that reflected by reservoir or field architecture, and reservoir compartmentalization. Properties may be used therein to obtain reservoir or field architecture or reservoir compartmentalization, compositional grading, and may be used to interpreted processes leading to various compositional grading or other equilibrium or disequilibrium distributions of fluids and fluid properties. Properties include, but are not limited to, the measured, calculated, and inferred properties obtained from sensor measurements and properties derived from interpretation, such as equation of state interpretation.

In certain embodiments, the methods and apparatus disclosed herein identify the phase of a dominant fluid for each channel. In certain embodiments, the methods and apparatus disclosed herein identify pure-phase channel observations versus mixed-phase channel observations. In certain embodiments, identifying the fluid type or fluid phase on a per-channel basis benefits one or more of: the estimation of fluid phase ratios or concentrations; the assessment of mud contamination; the construction of pure signature for the formation fluids; and the producible water cut of a zone including, but not limited to, a transition zone in which both formation oil and formation water is simultaneously sampled.

In certain embodiments, the method of fluid identification comprises clustering a plurality of channels to automatically classify an observed optical or non-optical spectrum into different fluid groups. In certain embodiments, the methods comprise fluid labeling of each of the fluid groups, wherein the fluid labeling is guided by the observation of a deterministic or probabilistic sensor channel that responds characteristically to different phases, e.g., a density sensor channel. In certain embodiments, a fluid ratio estimation and/or a fluid signature extraction are determined after completion of the fluid labeling step. Fluid ratio estimation and fluid signature estimation may be determined or extracted by grouping such as clustering and labeling fluids based on the characteristic channel observation including, but not limited to, the density observation.

Conventional methods may depend on pre-processing of the observed channel responses such as but not limited to optical data responses, such as debiasing and normalization. In contrast, the grouping methods such as clustering methods disclosed herein comprise a distribution, e.g., a statistical distribution, rather than exclusively an amplitude bias or scaling as in conventional methods. The grouping methods such as clustering methods disclosed herein present a more robust method for fluid identification. In certain embodiments, the fluid labeling methods disclosed herein improve fluid classification performance by sharing information between at least two paired channels of at least one sensor. Cross-sensor channel pairing is also possible. In certain embodiments, the fluid labeling methods disclosed herein improve the accuracy of channel pairs of low separability by importing guiding information, e.g., observed density, capacitance, resistivity, and/or acoustic information.

During formation tester pump-outs, reservoir fluids are often multi-phase flow including slug flow, dispersed flow, and emulsion flow, which may present difficulties in measuring combinations of liquids (water and oil) and gases or in some cases solids as well. It may be desirable to measure the physical and chemical properties of the individual phases of the fluids. The reservoir fluid compositions and distributions provide information for field engineers to make decisions on field development. Accurate gas composition may also assist in decision-making regarding the installation of expensive production facilities. Directly measuring the sensor responses, e.g., light-absorption responses for optical sensors of compositions in fluid samples, enables fluid identification, composition analysis, and physical and chemical properties analysis.

Properties of fluid samples may be measured either in a laboratory environment or in a real time subsurface borehole. Downhole fluid samples need not be captured in a container for analysis and, in certain embodiments, subsurface sensor channel measurements will be embodied by optical spectroscopy channels and a density sensor channel. Optical sensor channel analysis provides real-time information fluids at the field subsurface pressure and temperature. Other sensors with at least one channel include, but are not limited to, resistivity sensors, capacitance sensors, acoustic sensors, chromatographic sensors, microfluidic sensors, phase behavior sensors including compressibility sensors and bubble point sensors, electrochemical sensors, mass spectrometer and mass spectroscopy sensors. In certain embodiments, reservoir compositional variations are directly mapped with greater spatial resolution using downhole sampling compared to samples being sent to a laboratory. In certain embodiments, an in-situ compositional analysis is combined with a spatial mapping of compositional properties to provide an improved basis for selecting the locations from which to sample fluids for laboratory analysis. In certain embodiments, the sample quality is quantified as a aliquot representation of the formation fluid in the reservoir and contamination levels of drilling fluid filtrate as it is being withdrawn from a reservoir.

In some embodiments, a limited number of sensor channels, e.g., optical channels, is implemented in subsurface optical spectroscopy. For example, in certain embodiments, the optical spectra of fluid samples is measured channel-by-channel dynamically. In certain embodiments, multiple channels are acquired simultaneously at different locations. In certain embodiments, a viewing window of the channels oscillates between phases, or a combination of phases therein, and provides temporal analysis of the fluid's physical and chemical behavior.

In certain embodiments, a fluid's measured chemical behavior includes, but is not limited to, a petroleum composition comprising saturates, aromatics, resins, asphaltenes fractions, methane, ethane, propane, butane, pentane, hexane and higher components and individual or lumped higher hydrocarbon components (where lumping may be the composite analysis or reporting of two or more hydrocarbon components), inorganic component composition including water and nitrogen and carbon dioxide, and hydrogen sulfide chemical potential including reactive capability acidic levels of individual components, e.g., an organic acid, or as a whole, e.g., pH or total acid number (TAN) or redox potential. These chemical properties may be directly probed optically, by optical analysis in combination with other measurement devices, which may include, but may not be limited to, density, bubble point, compressibility, acoustic, NMR, capacitance, dielectric spectroscopy, nuclear methods, x-ray methods, terahertz methods, and resistivity.

In certain embodiments, chemical properties are interpreted based on physical, chemical, or empirical models as a secondary interpretation based on the directly probed chemical properties. For example, physical properties may include, but may not limited to, bubble point, compressibility, phase envelope, density, and viscosity, and may be measured directly by devices such as density sensors, viscometers, phase behavior experimentation, trapped volume devices, fractionation devices such as valved devices or membrane devices or derived by physical, chemical, or empirical models as a secondary interpretation based on directly probed physical properties. Physical properties may be measured or derived based, in part, on multiple measurements. As a non-limiting example for instance, phase behavior (or other physical properties), like compressibility or bubble point may be derived based on combinations of physical measurements and compositions as modeled by an equation of state (EOS) such as, but not limited to, as Peng Robertson or SRK cubic equation of state, a viral equation of state, or a PC-SAFT equation of state or an empirical machine learning model such as, but not limited to a neural network or a random forest model or a gradient boost method. Multiphase fluids provide difficulties for interpretation.

During a subsurface optical measurement, sampled formation fluids may include single-phase or multiphase mud contaminations. Alternatively, multiphase fluids may flow through the sampling path directly from multiphase formation fluids. Alternatively, multiphase fluids may be induced from phase changes due to pressure, volume, or temperature perturbations during sampling. In certain embodiments, sampled fluids for different channels are distributed in space or time, such as channels configured in a rotating wheel positioned in an optical path of a fluid phase detector wherein the fluids may be assumed to be the same phase (single-phase assumption). Consequently, obtaining the pure signature for the formation fluids and the mud filtrate may prove problematic, yielding errors for water/hydrocarbon ratio estimation and mud contamination assessment.

The present disclosure provides methods and apparatus for identifying the phases of dominating fluid for each channel, and further for identifying pure phase channel observations versus mixed phase channel observations. Identifying the fluid type on a per-channel basis further benefits the following: a) the estimation of fluid phase ratios or concentrations; b) the assessment of mud contamination; c) the construction of pure signature for the formation fluids; d) the producible water cut of a zone, including but not limited to, a transition zone; and e) the measurement of fluid properties for at least one of the inherent sample phases (oil, water, gas, solid).

In addition, the present disclosure provides methods and apparatus for performing downhole fluid analysis. Downhole fluid analysis based optical spectroscopy may be performed to distinguish between various water types (e.g., injected water, disposal water, formation water, mud filtrate) and to recognize ionic properties including ion concentrations. Furthermore, the methods and apparatus of the present disclosure may improve upon conventional practices by enabling diagnosis of water production and compatibility of a formation for salt water disposal and/or carbon capture and sequestration based on the analysis.

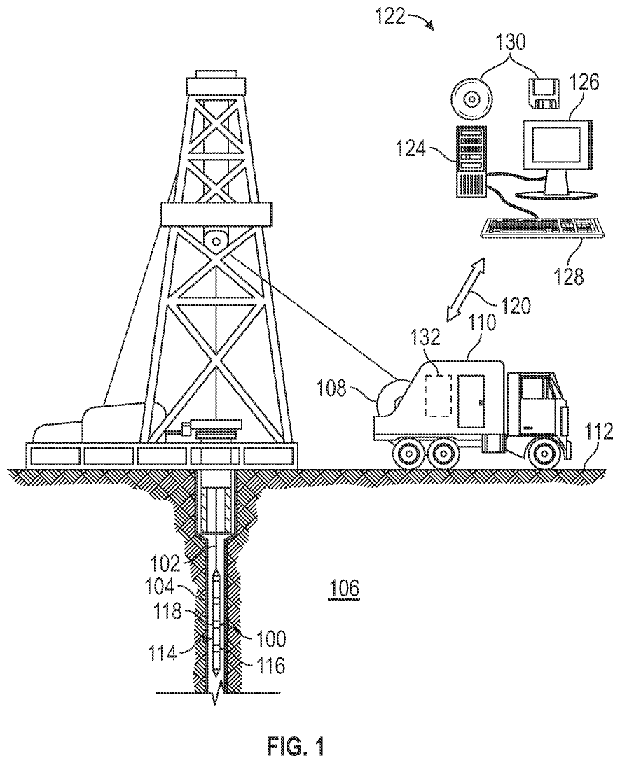

FIG. 1 is a schematic diagram of fluid sampling tool 100 on a conveyance 102. As illustrated, wellbore 104 may extend through subterranean formation 106. The reservoir fluid may be contaminated with well fluid, e.g., drilling fluid, from wellbore 104. As described herein, the fluid sample may be analyzed to determine fluid contamination and other fluid properties of the reservoir fluid. While the wellbore 104 is shown extending generally vertically into the subterranean formation 106, the principles described herein are also applicable to wellbores that extend at an angle through the subterranean formation 106, such as horizontal and slanted wellbores. For example, although FIG. 1 shows a vertical wellbore, an inclined wellbore or horizontal placement of the well and equipment is also possible. It should further be noted that while FIG. 1 generally depicts a land-based operation, those skilled in the art will readily recognize that the principles described herein are equally applicable to subsea operations that employ floating or sea-based platforms and rigs, without departing from the scope of the disclosure.

As illustrated, a hoist 108 may be used to run fluid sampling tool 100 into wellbore 104. Hoist 108 may be disposed on a vehicle 110. Hoist 108 may be used, for example, to raise and lower conveyance 102 in wellbore 104. While hoist 108 is shown on vehicle 110, it should be understood that conveyance 102 may alternatively be disposed from a hoist 108 that is installed at surface 112 instead of being located on vehicle 110. Fluid sampling tool 100 may be suspended in wellbore 104 on conveyance 102. Other conveyance types may be used for conveying fluid sampling tool 100 into wellbore 104, including coiled tubing and wired drill pipe, conventional drill pipe for example. Fluid sampling tool 100 may comprise a tool body 114, which may be elongated as shown on FIG. 1. Tool body 114 may be any suitable material, including without limitation titanium, stainless steel, alloys, plastic, combinations thereof, and the like. Fluid sampling tool 100 may further comprise one or more sensors 116 for measuring properties of the fluid sample, reservoir fluid, wellbore 104, subterranean formation 106, or the like. In examples, fluid sampling tool 100 may also include a fluid analysis module 118, which may be operable to process information regarding fluid sample, as described below. The fluid sampling tool 100 may be used to collect fluid samples from subterranean formation 106 and may obtain and separately store different fluid samples from subterranean formation 106.

In certain embodiments, fluid analysis module 118 comprises at least one sensor that is capable of continuously monitoring a reservoir fluid, e.g., optical sensors, acoustic sensors, electromagnetic sensors, conductivity sensors, resistivity sensors, a capacitance sensor, selective electrodes, density sensors, mass sensors, thermal sensors, chromatography sensors, viscosity sensors, bubble point sensors, fluid compressibility sensors, flow rate sensors. Sensors may measure a contrast between drilling fluid filtrate properties and formation fluid properties. Fluid analysis module 118 may be operable to derive properties and characterize the fluid sample. By way of example, fluid analysis module 118 may measure absorption, transmittance, or reflectance spectra and translate such measurements into component concentrations of the fluid sample, which may be lumped component concentrations, as described above. The fluid analysis module 118 may also measure gas-to-oil ratio, fluid composition, water cut, live fluid density, live fluid viscosity, formation pressure, and formation temperature. Fluid analysis module 118 may also be operable to determine fluid contamination of the fluid sample and may include any instrumentality or aggregate of instrumentalities operable to compute, classify, process, transmit, receive, retrieve, originate, switch, store, display, manifest, detect, record, reproduce, handle, or utilize any form of information, intelligence, or data for business, scientific, control, or other purposes. In certain embodiments, the fluid analysis module 118 includes one or more of a random access memory (RAM), a processing unit such as a central processing unit (CPU), a read-only memory (ROM) and/or other types of nonvolatile memory.

Any suitable technique may be used for transmitting phase signals from the fluid sampling tool 100 to the surface 112. As illustrated, a communication link 120 (which may be wired or wireless, for example) may be provided that may transmit data from fluid sampling tool 100 to an information handling system 122 at surface 112. Information handling system 122 may include a processing unit 124, a monitor 126, an input device 128 (e.g., keyboard, mouse, etc.), and/or computer media 130 (e.g., optical disks, magnetic disks) that can store code representative of the methods described herein. The information handling system 122 may act as a data acquisition system and possibly a data processing system that analyzes information from fluid sampling tool 100. For example, information handling system 122 may process the information from fluid sampling tool 100 for determination of fluid contamination. The information handling system 122 may also determine additional properties of the fluid sample (or reservoir fluid), such as component concentrations, pressure-volume-temperature properties (e.g., bubble point, phase envelop prediction, etc.) based on the fluid characterization. In certain embodiments, the processing occurs in real-time at surface 112. In certain embodiments, the processing occurs downhole or another location after recovery of fluid sampling tool 100 from wellbore 104. In certain embodiments, the processing is performed by an information handling system in wellbore 104, e.g., fluid analysis module 118 and the measured fluid properties are then transmitted to surface 112.

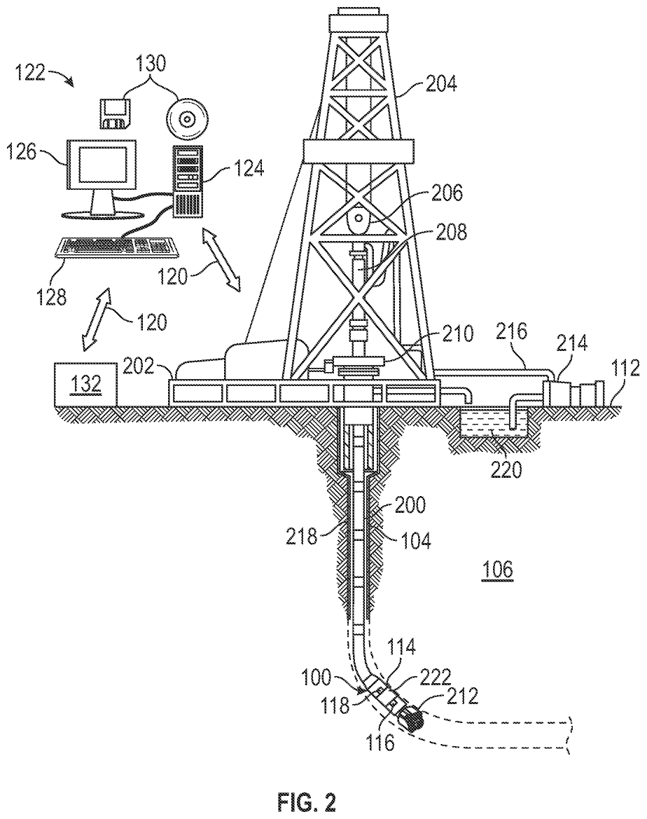

FIG. 2 is a schematic diagram of an example fluid sampling tool 100 disposed on a drill string 200 in a drilling operation. Fluid sampling tool 100 is used to obtain a fluid sample, for example, a fluid sample of a reservoir fluid from subterranean formation 106. The reservoir fluid may be contaminated with well fluid (e.g., drilling fluid) from wellbore 104. As described herein, the fluid sample may be analyzed to determine fluid contamination and other fluid properties of the reservoir fluid. As illustrated, a wellbore 104 may extend through subterranean formation 106. Drilling platform 202 supports a derrick 204 having a traveling block 206 for raising and lowering drill string 200. A kelly 208 supports drill string 200 as it is lowered through a rotary table 210. Drill bit 212 is attached to the distal end of drill string 200 and is driven either by a downhole motor and/or via rotation of drill string 200 from the surface 112. Drill bit 212 may comprise, roller cone bits, PDC bits, natural diamond bits, any hole openers, reamers, coring bits, and the like. As drill bit 212 rotates, it creates and extends wellbore 104 through various subterranean formations 106. A pump 214 circulates a drilling fluid through a feed pipe 216 to kelly 208, downhole through interior of drill string 200, through orifices in drill bit 212, back to surface 112 via annulus 218 surrounding drill string 200, and into a retention pit 220.

Drill bit 212 may be just one piece of a downhole assembly that includes one or more drill collars 222 and fluid sampling tool 100 that gathers measurements and fluid samples. In certain embodiments, fluid sampling tool 100 is built into a drill collar 222. One or more of the drill collars 222 may form a tool body 114, which may be elongated as shown on FIG. 2. Fluid sampling tool 100 may be similar in configuration and operation to fluid sampling tool 100 shown on FIG. 1 except that FIG. 2 shows fluid sampling tool 100 disposed on drill string 200. In certain embodiments, the sampling tool 100 may be lowered into the wellbore after drilling operations on a wireline.

The properties of the sampled fluid are measured as the fluid passes from the formation through the tool and into either the wellbore or a sample container. As fluid is flushed in the near wellbore region by the mechanical pump, the fluid that passes through the tool generally reduces in drilling fluid filtrate content, and generally increases in formation fluid content. In certain embodiments, the fluid sampling tool 100 is used to collect a fluid sample from subterranean formation 106 when the filtrate content has been determined to be sufficiently low. In certain embodiments, e.g., laboratory testing, drilling fluid contamination below 10% is sufficiently low. In certain embodiments, e.g., drilling fluid filtrate contamination testing, below 1% is sufficiently low. In certain embodiments, lower drilling fluid contamination requirements are needed, e.g., a lighter oil as designated with either a higher GOR or a higher API gravity. In certain embodiments, the limit on drilling fluid contamination depend on the rate of cleanup in a cost-benefit analysis, since longer pump-out times utilized to incrementally reduce the contamination levels have a larger cost.

In certain embodiments, fluid sampling tool 100 separately stores different fluid samples from subterranean formation 106 with fluid analysis module 118. In certain embodiments, the storage of the fluid samples in the fluid sampling tool 100 is based on the determination of the fluid contamination. For example, if the fluid contamination in a fluid sample exceeds a threshold then the fluid sample is not stored otherwise the fluid sample is stored in fluid sampling tool 100.

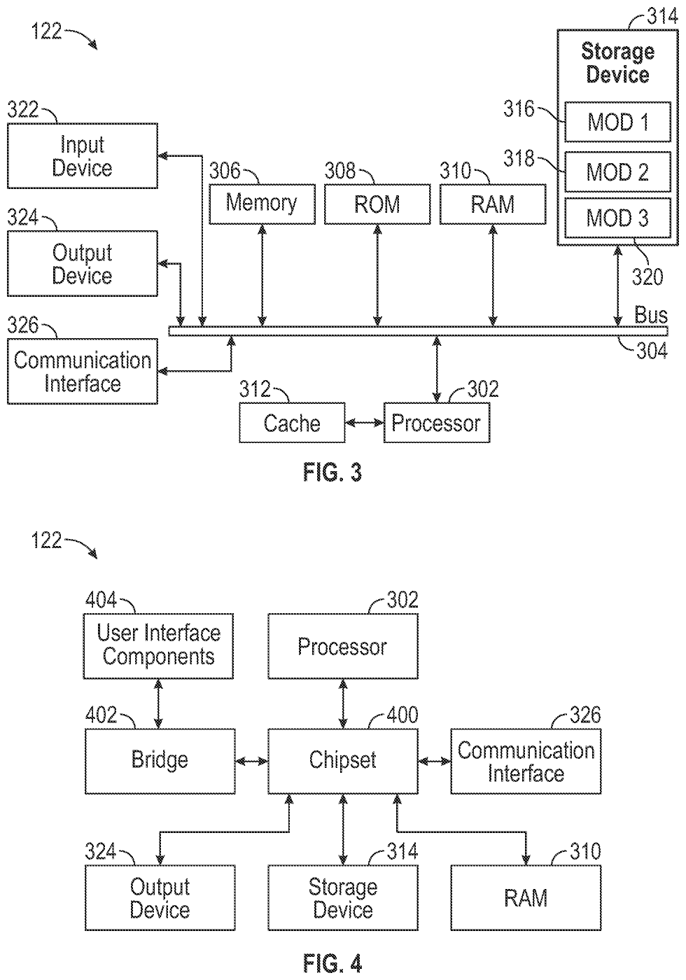

FIG. 3 illustrates an example information handling system 122 employed to perform various blocks, methods, and techniques disclosed herein. As illustrated, information handling system 122 includes a processing unit (CPU or processor) 302 and a system bus 304 that couples various system components including system memory 306 such as read only memory (ROM) 308 and random-access memory (RAM) 310 to processor 302. Processors disclosed herein may all be forms of this processor 302. Information handling system 122 may include a cache 312 of high-speed memory connected directly with, in close proximity to, or integrated as part of processor 302. Information handling system 122 copies data from memory 306 and/or storage device 314 to cache 312 for quick access by processor 302. In this way, cache 312 provides a performance boost that avoids processor 302 delays while waiting for data. These and other modules may control or be configured to control processor 302 to perform various operations or actions. Other system memory 306 may be available for use as well. Memory 306 may include multiple different types of memory with different performance characteristics. It may be appreciated that the disclosure may operate on information handling system 122 with more than one processor 302 or on a group or cluster of computing devices networked together to provide greater processing capability. Processor 302 may include any general-purpose processor and a hardware module or software module, such as first module 316, second module 318, and third module 320 stored in storage device 314, configured to control processor 302 as well as a special-purpose processor where software instructions are incorporated into processor 302. Processor 302 may be a self-contained computing system, containing multiple cores or processors, a bus, memory controller, cache, etc. A multi-core processor may be symmetric or asymmetric. Processor 302 may include multiple processors, such as a system having multiple, physically separate processors in different sockets, or a system having multiple processor cores on a single physical chip. Similarly, processor 302 may include multiple distributed processors located in multiple separate computing devices but working together such as via a communications network. Multiple processors or processor cores may share resources such as memory 306 or cache 312 or may operate using independent resources. Processor 302 may include one or more state machines, an application specific integrated circuit (ASIC), or a programmable gate array (PGA) including a field PGA (FPGA).

Each individual component discussed above may be coupled to system bus 304, which may connect each and every individual component to each other. System bus 304 may be any of several types of bus structures including a memory bus or memory controller, a peripheral bus, and a local bus using any of a variety of bus architectures. A basic input/output (BIOS) stored in ROM 308 or the like, may provide the basic routine that helps to transfer information between elements within information handling system 122, such as during start-up. Information handling system 122 further includes storage devices 314 or computer-readable storage media such as a hard disk drive, a magnetic disk drive, an optical disk drive, tape drive, solid-state drive, RAM drive, removable storage devices, a redundant array of inexpensive disks (RAID), hybrid storage device, or the like. Storage device 314 may include software modules 316, 318, and 320 for controlling processor 302. Information handling system 122 may include other hardware or software modules. Storage device 314 is connected to the system bus 304 by a drive interface. The drives and the associated computer-readable storage devices provide nonvolatile storage of computer-readable instructions, data structures, program modules and other data for information handling system 122. In one aspect, a hardware module that performs a particular function includes the software component stored in a tangible computer-readable storage device in connection with the necessary hardware components, such as processor 302, system bus 304, and so forth, to carry out a particular function. In another aspect, the system may use a processor and machine-readable storage device to store instructions which, when executed by the processor, cause the processor to perform operations, a method or other specific actions. The basic components and appropriate variations may be modified depending on the type of device, such as whether information handling system 122 is a small, handheld computing device, a desktop computer, or a computer server. When processor 302 executes instructions to perform “operations,” processor 302 may perform the operations directly and/or facilitate, direct, or cooperate with another device or component to perform the operations.

As illustrated, information handling system 122 employs storage device 314, which may be a hard disk or other types of computer-readable storage devices which may store data that are accessible by a computer, such as magnetic cassettes, flash memory cards, digital versatile disks (DVDs), cartridges, random access memories (RAMs) 310, read only memory (ROM) 308, a cable containing a bit stream and the like, may also be used in the exemplary operating environment. Tangible machine-readable storage media, computer-readable storage devices, or computer-readable memory devices, expressly exclude media such as transitory waves, energy, carrier signals, and EM waves.

To enable user interaction with information handling system 122, an input device 322 represents any number of input mechanisms, such as a microphone for speech, a touch-sensitive screen for gesture or graphical input, keyboard, mouse, motion input, speech and so forth. Additionally, input device 322 may receive acoustic or EM measurements from fluid sampling tool 100 (e.g., referring to FIGS. 1 and 2), discussed above. An output device 324 may also be one or more of a number of output mechanisms known to those of skill in the art. In some instances, multimodal systems enable a user to provide multiple types of input to communicate with information handling system 122. Communications interface 326 generally governs and manages the user input and system output. There is no restriction on operating on any particular hardware arrangement and therefore the basic hardware depicted may easily be substituted for improved hardware or firmware arrangements as they are developed.

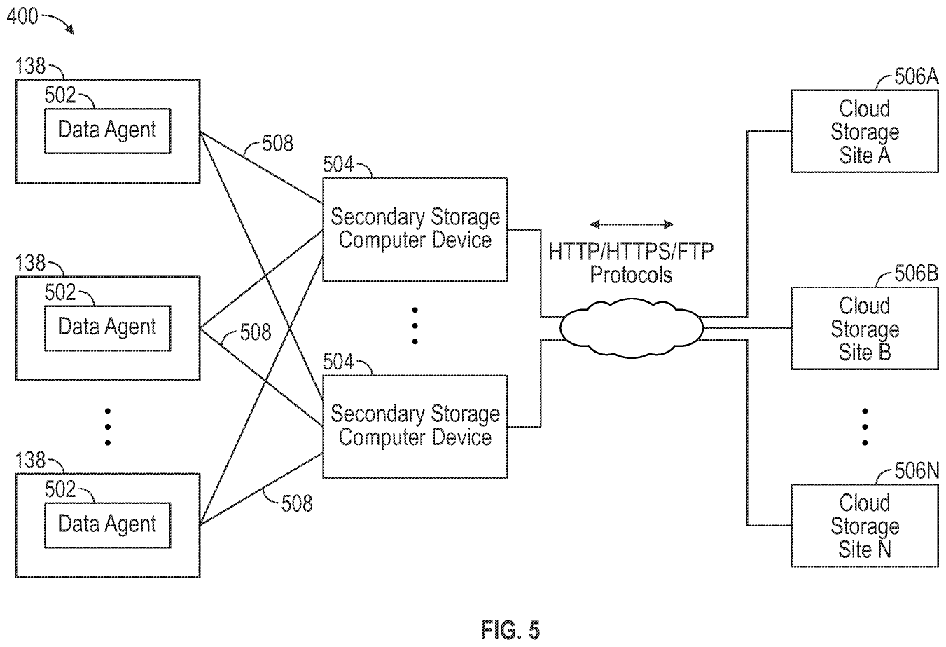

As illustrated, each individual component is depicted and disclosed as individual functional blocks. The functions these blocks represent may be provided through the use of either shared or dedicated hardware, including, but not limited to, hardware capable of executing software and hardware, such as a processor 302, that is purpose-built to operate as an equivalent to software executing on a general-purpose processor. For example, the functions of one or more processors presented in FIG. 5 may be provided by a single shared processor or multiple processors. The term “processor” shall not be construed to refer exclusively to hardware capable of executing software. Illustrative embodiments may include microprocessor and/or digital signal processor (DSP) hardware, read-only memory (ROM) 308 for storing software performing the operations described below, and random-access memory (RAM) 310 for storing results. Very large-scale integration (VLSI) hardware embodiments, as well as custom VLSI circuitry in combination with a general-purpose DSP circuit, may also be provided.

FIG. 4 illustrates an example information handling system 122 having a chipset architecture for information handling system 122 that may be used in executing the described method and generating and displaying a graphical user interface (GUI). Information handling system 122 is an example of computer hardware, software, and firmware that may be used to implement the disclosed technology. Information handling system 122 may include a processor 302, representative of any number of physically and/or logically distinct resources capable of executing software, firmware, and hardware configured to perform identified computations. Processor 302 may communicate with a chipset 400, discussed below, that may control input to and output from processor 302. In this example, chipset 400 outputs information to output device 324, such as a display, and may read and write information to storage device 314, which may include, for example, magnetic media, and solid-state media. Chipset 400 may also read data from and write data to RAM 310. A bridge 402 for interfacing with a variety of user interface components 504 may be provided for interfacing with chipset 400. Such user interface components 404 may include a keyboard, a microphone, touch detection and processing circuitry, a pointing device, such as a mouse, and so on. In general, inputs to information handling system 122 may come from any of a variety of sources, machine generated and/or human generated.

In certain embodiments, chipset 400 interfaces with one or more communication interfaces 326 that may have different physical interfaces. Such communication interfaces include interfaces for wired and wireless local area networks, for broadband wireless networks, as well as personal area networks. Some embodiments of the methods for generating, displaying, and using the GUI disclosed herein include receiving ordered datasets over the physical interface or be generated by the machine itself by processor 302 analyzing data stored in storage device 314 or RAM 310. Further, information handling system 122 receive inputs from a user via user interface components 404 and execute appropriate functions, such as browsing functions by interpreting these inputs using processor 302.

Computer-executable instructions include, for example, instructions and data which cause a general-purpose computer, special purpose computer, or special purpose processing device to perform a certain function or group of functions. Computer-executable instructions also include program modules that are executed by computers in stand-alone or network environments. Generally, program modules include routines, programs, components, data structures, objects, and the functions inherent in the design of special-purpose processors, etc. that perform particular tasks or implement particular abstract data types. Computer-executable instructions, associated data structures, and program modules represent examples of the program code means for executing blocks of the methods disclosed herein. The particular sequence of such executable instructions or associated data structures represents examples of corresponding acts for implementing the functions described in such blocks.

In additional examples, the disclosed methods may be practiced in network computing environments with many types of computer system configurations, including personal computers, hand-held devices, multi-processor systems, microprocessor-based or programmable consumer electronics, network PCs, minicomputers, mainframe computers, and the like. Examples may also be practiced in distributed computing environments where tasks are performed by local and remote processing devices that are linked (either by hardwired links, wireless links, or by a combination thereof) through a communications network. In a distributed computing environment, program modules may be located in both local and remote memory storage devices.

FIG. 5 illustrates an example of one arrangement of resources on a computing network 500 that employs the processes and techniques described herein. As noted above, an information handling system 122 utilizes data, which includes files, databases, directories, metadata (e.g., access control list (ACLS) creation/edit dates associated with the data, etc.), and other data objects. The data on the information handling system 122 is typically a primary copy (e.g., a production copy). During a copy, backup, archive or other storage operation, information handling system 122 may send a copy of some data objects (or some components thereof) to a secondary storage computing device 504 by utilizing one or more data agents 502.

In certain embodiments, a data agent 502 is one of a desktop application, website application, or any software-based application that is run on information handling system 122. In certain embodiments, the information handling system 122 is disposed at one of a rig site, e.g., as shown in FIG. 1, off site location, core laboratory, repair and manufacturing center, and/or the like. In certain embodiments, the data agent 502 communicates with a secondary storage computing device 504 using communication protocol 508 in a wired or wireless system. Communication protocol 508 may function and operate as an input to a website application. In the website application, field data related to pre- and post-operations, generated DTCs, notes, and/or the like may be uploaded. Additionally, information handling system 122 may utilize communication protocol 508 to access processed measurements, operations with similar DTCs, troubleshooting findings, historical run data, and/or the like. This information is accessed from secondary storage computing device 504 by data agent 502, which is loaded on information handling system 122.

Secondary storage computing device 504 may operate and function to create secondary copies of primary data objects (or some components thereof) in various cloud storage sites 506A-N. Additionally, secondary storage computing device 504 may run determinative algorithms on data uploaded from one or more information handling systems 122, discussed further below. Communications between the secondary storage computing devices 504 and cloud storage sites 506 AN may utilize REST protocols (Representational state transfer interfaces) that satisfy basic C/R/U/D semantics (Create/Read/Update/Delete semantics), or other hypertext transfer protocol (“HTTP”)-based or file-transfer protocol (“FTP”)-based protocols (e.g., Simple Object Access Protocol).

In conjunction with creating secondary copies in cloud storage sites 506A-N, the secondary storage computing device 504 may also perform local content indexing and/or local object-level, subobject-level or block-level deduplication when performing storage operations involving various cloud storage sites 506A-N. Cloud storage sites 506A-N may further record and maintain, EM logs, map DTC codes, store repair and maintenance data, store operational data, and/or provide outputs from determinative algorithms that are located in cloud storage sites 506A-N. In a non-limiting example, this type of network may be utilized as a platform to store, backup, analyze, import, preform extract, transform and load (“ETL”) processes, mathematically process, apply machine learning models, and augment data sets.

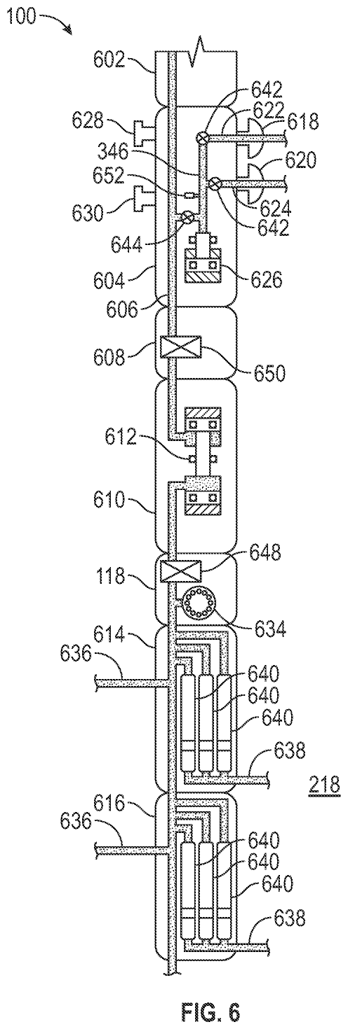

FIG. 6 is a schematic of an example fluid sampling tool 100. In certain embodiments, the fluid sampling tool 100 includes a power telemetry section 602 through which the tool communicates with other actuators and sensors 116 in drill string 200 or conveyance 102 (e.g., referring to FIGS. 1 and 2), the drill string's telemetry section 602, and/or directly with a surface telemetry system (not illustrated). In certain embodiments, power telemetry section 602 is a port through which the various actuators (e.g., valves) and sensors (e.g., temperature and pressure sensors) in the fluid sampling tool 100 are controlled and monitored. In certain embodiments, power telemetry section 602 includes a computer that exercises the control and monitoring function. In certain embodiments, the control and monitoring function is performed by a computer in another part of the drill string or wireline tool (not shown) or by information handling system 122 on surface 112.

In certain embodiments, the fluid sampling tool 100 includes a dual probe section 604 that extracts fluid from the reservoir and delivers it to a passageway 606 that extends from one end of fluid sampling tool 100 to the other. In this example, the dual probe section 604 includes two probes 618, 620 that extend from fluid sampling tool 100 and press against the inner wall of wellbore 104 (e.g., referring to FIG. 1). Probe channels 622, 624 connect probes 618, 620 to passageway 606. The high-volume bidirectional pump 612 is used to pump fluids from the reservoir, through probe channels 622, 624 and to passageway 606. In certain embodiments, a low volume pump 626 is used for this purpose. Two standoffs or stabilizers 628, 630 hold fluid sampling tool 100 in place as probes 318, 320 press against the wall of wellbore 104. In certain embodiments, probes 618, 620 and stabilizers 628, 630 may be retracted when the fluid sampling tool 100 is in motion and extended to sample the formation fluids at any suitable location in wellbore 104. In certain embodiments, other probe sections include focused sampling probes, oval probes, an/or packers.

In certain embodiments, passageway 606 is connected to other tools disposed on drill string 200 or conveyance 102 (e.g., referring to FIGS. 1 and 2). In certain embodiments, fluid sampling tool 100 includes a quartz gauge section 608, that includes one or more sensors to allow measurement of properties, e.g., temperature and pressure, of fluid in passageway 606. In certain embodiments, the fluid sampling tool 100 includes a flow-control pump-out section 610 with a high-volume bidirectional pump 612 for pumping fluid through passageway 606. In certain embodiments, fluid sampling tool 100 includes two multi-chamber sections 614, 616, referred to collectively as multi-chamber sections 614, 616 or individually as first multi-chamber section 614 and second multi-chamber section 616, respectively.

In certain embodiments, multi-chamber sections 614, 616 are separated from flow-control pump-out section 610 by sensor section 632, which houses at least one non-optical fluid sensor 648, 650 and/or at least optical measurement tool 634. It should be noted that non-optical fluid sensor 648, 650 and optical measurement tool 634 may be disposed in any order on passageway 606. Additionally, although depicted in sensor section 632, the non-optical fluid sensors 648, 650 and optical measurement tool 334 may be disposed along passageway 306 at any suitable location within fluid sampling tool 100.

Non-optical fluid sensor 648, 650 may be displaced within sensor section 632 in-line with passageway 606 to be a “flow through” sensor. In certain embodiments, non-optical fluid sensor 648, 650 are connected to passageway 606 via an offshoot of passageway 606. In certain embodiments, optical measurement tool 634 includes one or more of a density sensor, a capacitance sensor, a resistivity sensor, and/or combinations thereof. In certain embodiments, non-optical fluid sensor 648, 650 operates to measure fluid properties of drilling fluid filtrate.

In certain embodiments, optical measurement tool 634 is displaced within sensor section 632 in-line with passageway 606 to be a “flow through” sensor. In certain embodiments, optical measurement tool 634 is connected to passageway 606 via an offshoot of passageway 606. In certain embodiments, optical measurement tool 634 includes one or more of an optical sensor, an acoustic sensor, an electromagnetic sensor, a conductivity sensors, a resistivity sensor, a capacitance sensor, selective electrodes, a density sensors, mass sensors, thermal sensors, chromatography sensors, viscosity sensors, bubble point sensor, a fluid compressibility sensor, a flow rate sensor, a microfluidic sensor, a selective electrode such as an ion selective electrode, and/or combinations thereof. In certain embodiments, optical measurement tool 634 operates to measure drilling fluid filtrate, discussed further below.

In certain embodiments, multi-chamber section 614, 616 comprises an access channel 636 and a chamber access channel 638. In certain embodiments, access channel 636 and chamber access channel 638 operate to either allow a solids-containing fluid (e.g., mud) disposed in wellbore 104 in or provide a path for removing fluid from fluid sampling tool 100 into wellbore 104. As illustrated, multi-chamber section 614, 616 comprises a plurality of chambers 640. Chambers 640 may be used to sample wellbore fluids, formation fluids, and/or the like during measurement operations.

In certain embodiments, a pump-out operation is performed during downhole measurement operations. A pump-out is an operation where at least a portion of a fluid which may contain solids, e.g., drilling fluid, and may move through fluid sampling tool 100 until substantially increasing concentrations of formation fluids enter fluid sampling tool 100. For example, during pump-out operations, probes 618, 620 may be pressed against the inner wall of wellbore 104 (e.g., referring to FIG. 1). Pressure may increase at probes 618, 620 due to compression against the formation 106 (e.g., referring to FIG. 1 or 2) exerting pressure on probes 618, 620. As pressure rises and reaches a predetermined pressure, valves 642 opens so as to close equalizer valve 644, thereby isolating fluid passageway 646 from annulus 218. In this manner, valve 642 ensures that equalizer valve 644 closes only after probes 618, 620 has entered contact with mud cake (not illustrated) that is disposed against the inner wall of wellbore 104. In certain embodiments, with probes 618, 620 pressed against the inner wall of wellbore 104, the pressure rises and closes the equalizer valve in fluid passageway 646, thereby isolating the fluid passageway 646 from the annulus 218. In this manner, the equalizer valve in fluid passageway 646 closes before probes 618, 620 have entered contact with the mud cake that lines the inner wall of wellbore 104. Fluid passageway 646, now closed to annulus 218, is in fluid communication with low volume pump 626.

As low volume pump 626 is actuated, formation fluid is drawn through probe channels 622, 624 and probes 618, 620. The movement of low volume pump 626 lowers the pressure in fluid passageway 646 to a pressure below the formation pressure, such that formation fluid is drawn through probe channels 622, 624 and probes 618, 620 and into fluid passageway 646. Probes 618, 620 serves as a seal to prevent annular fluids from entering fluid passageway 646. Such an operation as described may take place before, after, during or as part of a sampling operation.

With low volume pump 626 in its fully retracted position and formation fluid drawn into fluid passageway 646, the pressure stabilizes and enables pressure sensor 652 to sense and measure formation fluid pressure. The measured pressure is transmitted to information handling system 122 disposed on formation testing tool 100 and/or it may be transmitted to the surface via mud pulse telemetry or by any other conventional telemetry means to an information handling system 122 disposed on surface 112. During this interval, pressure sensor 652 may continuously monitor the pressure in fluid passageway 646 until the pressure stabilizes, or after a predetermined time interval. When the measured pressure stabilizes, e.g., at 1800 psi, or after a predetermined time interval, the drawdown operation may be complete.

Next, high-volume bidirectional pump 612 activates and equalizer valve 644 is opened. This allows for formation fluid to move toward high-volume bidirectional pump 612 through passageway 606. Formation fluid moves through passageway 606 to sensor section 632. Once the drilling fluid filtrate has moved into sensor section 632 high-volume bidirectional pump 612 stops. This allows the drilling fluid filtrate to be measured by optical measurement tool 634 within sensor section 632.

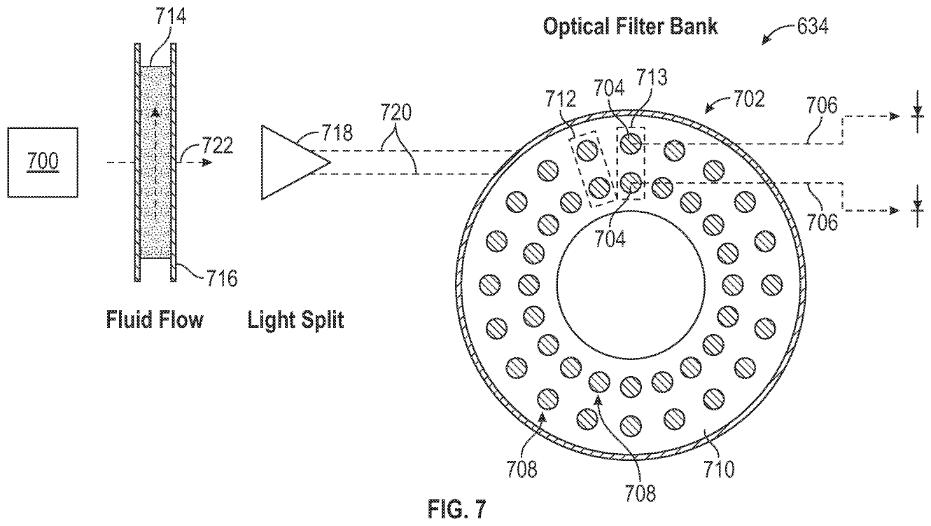

FIG. 7 depicts an example hardware configuration of a dynamic subsurface optical measurement tool 334. Optical measurement tool 634 includes a light source 700, a filter bank 702 comprising a plurality of optical filters 704 (measurement of the light transmittance through an optical filter 704 is called a channel 706) configured as two rings 708 on optical plate 710, within a channel pair 712 on each azimuth. In certain embodiments, one or more channels 706 comprise a different optical filter 704 to measured different properties of fluid sample 714. During the rotation of optical plate 710, the two optical filters 704 on a channel pair 712 may be synchronized spatially or temporally to measure substantially the same fluid sample 714 in viewing area 716, which may be a glass pipe. An active channel pair 713 is a channel pair 712 in which optical measurements are being taken to form one or more channels 706. In certain embodiments, channel pairs 712 are synchronized such that channel pairs 712 have a sufficient probability of observing the same phase, i.e., better than 10% but more desirably more than 50% and yet more desirably more than 80%. In certain embodiments, more than two channels 706 may be sufficiently synchronized according to a desired probability of observing a single phase in time or space. In certain embodiments, a velocity calculation of the fluid phase specific velocities is used to aid synchronization over distance or time. In certain embodiments, distribution or autocorrelation calculations are used to improve the synchronization over longer distances or time. During measurement operations, fluid samples 714, which is formation fluid from passageway 606, flows through a viewing region, e.g., a transparent region of the flow path. In certain embodiments, the viewing region or viewing area is not be transparent to visible light but rather to the form of energy used to measure the fluid characteristics for a given sensor. In certain embodiments, a viewing region for an acoustic sensor ideally has a low acoustic impedance. In certain embodiments, the viewing region is transparent, i.e., passes energy with low attenuation, to infrared light. In certain embodiments, the viewing region is transparent to magnetic fields. In certain embodiments, the viewing area 716 is more generally a measurement region, e.g., for a phase behavior sensors or a density sensor. In certain embodiments, the viewing region is part of passageway 606 and/or a branch off of passageway 606). In certain embodiments, the viewing region is transparent and light 722 absorbed by fluid sample 714 may be split through a prism 718 into at least two ray paths 720. Split light rays 720 is measured by a detector (not shown as they pass through channel pair 712. In certain embodiments, filter bank 702 rotates to another channel pair 712 after the measurement of each channel 706 from channel pair 712 and dynamically gathers an optical spectra measurement of all channels after a full sampling channel rotation. In certain embodiments, the optical filter bank 634 comprises one or more optical filters 704, one or more filter banks 702, and one or more channels 706.

As described, optical measurement tool 634 may be used in a downhole environment to perform measurements on fluid samples 714. Analysis of collected data may occur at various locations in a system or at various steps in a method in accordance with the present disclosure. For example, processing of the collected data may occur at any suitable location including, without limitation, at fluid analysis module 118 and/or information handling system 122.

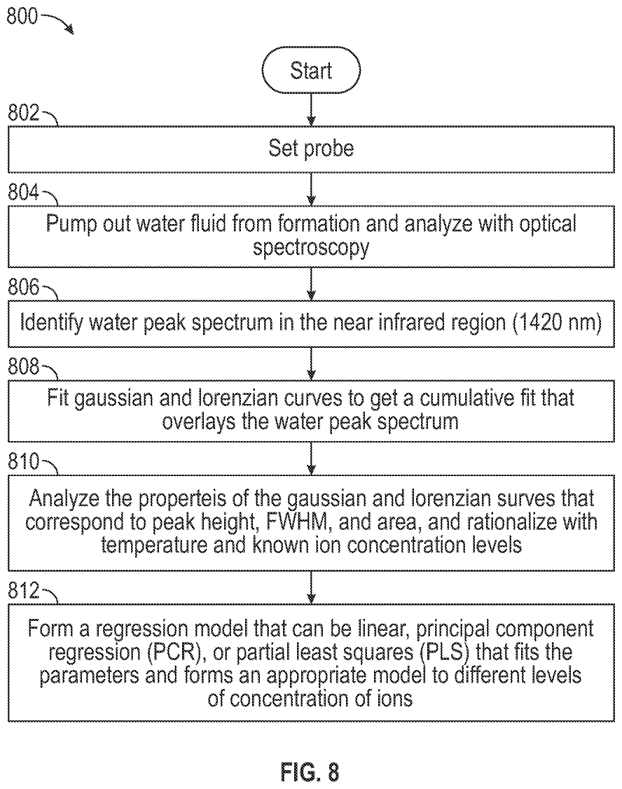

FIG. 8 illustrates an example workflow 800 to determine a concentration of ions. In certain embodiments, the workflow 800 is performed on information handling system 122. Within this disclosure, the term “ions” means one or more of sodium, potassium, calcium, magnesium, manganese, strontium, barium, iron (ii), iron (iii), chromium (ii), chromium (iii), copper (i), copper (ii), fluoride, bromide, iodide, carbonate, oxalate, nitrite, nitrate, phosphite, phosphate, sulfite, sulfate, hydrogen carbonate, hydrogen sulfite (“bisulfite”), hydrogen sulfate (“bisulfate”), hydronium, hydrogen phosphate, dihydrogen phosphate, chromate, dichromate, acetate, thiosulfate, hydride, lithium, zinc, ammonium, cyanide, permanganate, mercury (i), mercury (ii), and combinations thereof. Workflow 800 begins in block 802 where the fluid sampling tool 100 is deployed downhole. In certain embodiments, deployment comprises pressing the dual probe section 604 of fluid sampling tool 100 against the inner wall of wellbore 104. Workflow 800 proceed to block 804, wherein a pump-out operation occurs. In block 804, fluid sampling tool 100 is used to pump out fluid from the formation and analyze a sample with optical spectroscopy, e.g., using optical measurement tool 634 and fluid analysis module 118. In block 806, fluid analysis module 118 and/or information handling system 122 are used to interpret the information gathered by the optical measurement tool 634 and identify a water peak spectrum in the near infrared region, e.g., in the wavelength region around 1420 nm. In block 808, a Gaussian and/or a Lorenzian curve are fit to the data to get a cumulative fit that overlays the water peak spectrum. In block 810, fluid analysis module 118 or information handling system 122 may analyze the properties of the Gaussian and Lorenzian curves that correspond to peak height, FWHM, and area, and rationalize with temperature and known ion concentration levels. In block 812, a regression model is formed that can be linear, principal component regression (PCR), or partial least squares (PLS) that fits the parameters and forms an appropriate model to different levels of concentration of ions.

In certain embodiments, the model formed in block 812 is used to carry out a variety of executable instructions, e.g., distinguishing filtrate from water. In certain embodiments, the model is used to show unique characteristics between waters in-situ. In certain embodiments, the model uses inputs of one or more parameters and yields as outputs one or more parameters.

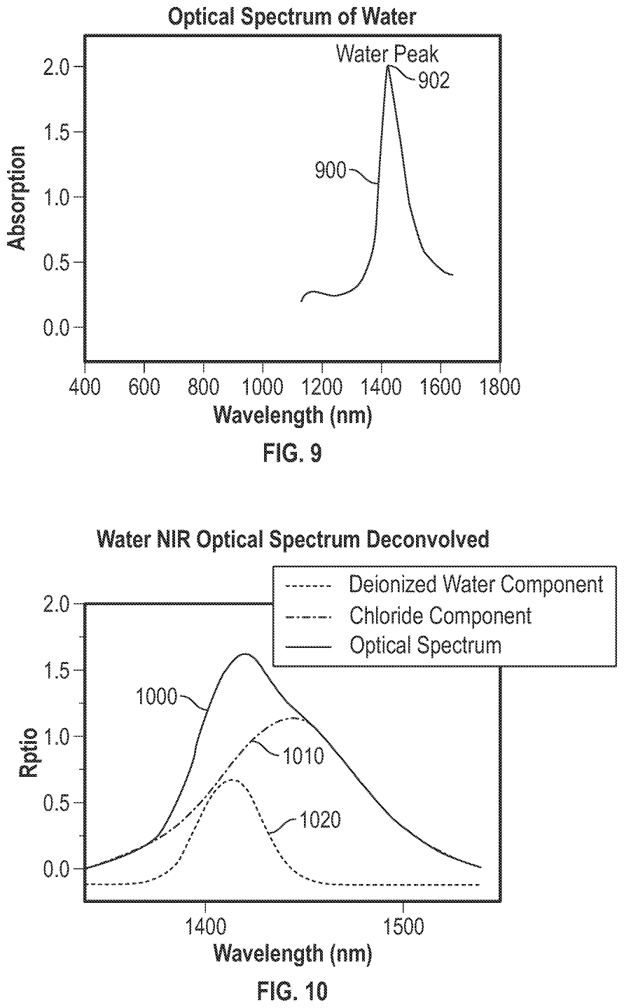

FIG. 9 is a graph illustrating an example of “fingerprinting” a fluid, e.g., to differentiate between injected fluid and formation water. Water peak 902 is an absorption peak as a function of wavelength that is characteristic of pure water. In certain embodiments, optical spectroscopy is performed on other components commonly present in formation/wellbore fluids and the results thereof may be compiled, e.g., a library, for reference. Interpretation of measurement data may assume a single ion in a sample or multiple ions. In embodiments where multiple ion concentrations are assumed, such as for samples comprising a plurality of water types or a mixture of fluids from a plurality of water sources, accuracy of a model may be optimized using fingerprints of the various types.

FIG. 10 is a graph illustrating an example of deconvolving an optical measurement into components. The measurement data of this example comprises synthetic data representative of data collected from a downhole environment. The solid line 1000 represents an overall optical spectrum representative of results of optical spectroscopy of a fluid sample. This data is deconvolved into a dot-dash line 1010 of an ion component, e.g., chloride, using its fingerprint, and a residual component indicated by the dotted line 1020 that can be identified as deionized water component using a predetermined fingerprint library.

Models in accordance with the present disclosure may be developed experimentally to determine ion concentrations at varying fluid salinities, temperatures, and/or other conditions. These experimentally derived models may distinguish a mud filtrate from water as well as between waters.

### Deterministic Method

A deterministic method for analyzing contamination in formation fluids offers several advantages over conventional empirical, trend-fitting methods. Firstly, deterministic methods provide a clear, mechanistic understanding of the contamination process, which can lead to more accurate and reliable estimates of contamination levels. They allow for the direct calculation of contamination based on measured properties of the fluid, eliminating the need for trend fitting or statistical analysis. This reduces the uncertainty associated with these methods and provides more precise estimates of formation fluid properties. In certain embodiments, deterministic methods provide real-time feedback during the pump-out process, thereby enabling immediate adjustments to minimize contamination. They also allow for the prediction of contamination levels under different conditions, which can be invaluable for planning and optimizing formation testing operations. Overall, a deterministic approach provides a robust and reliable framework for contamination estimation, leading to improved formation fluid sampling and analysis. The primary challenge to a deterministic method is a lack of understanding of the properties of the formation fluid and the present embodiment proposes a method to overcome this challenge.

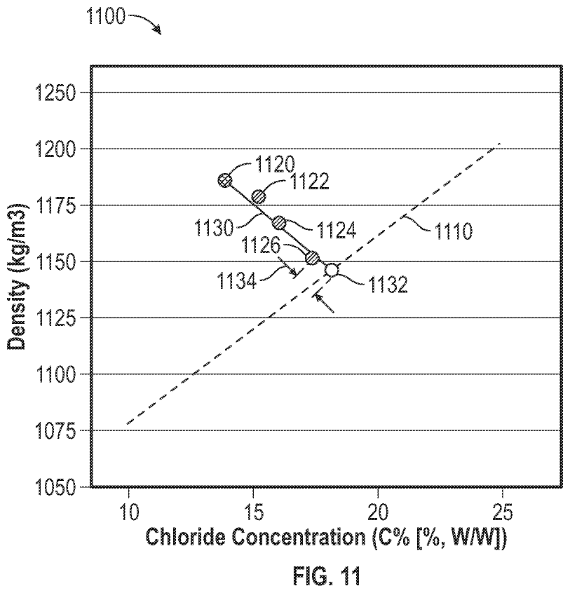

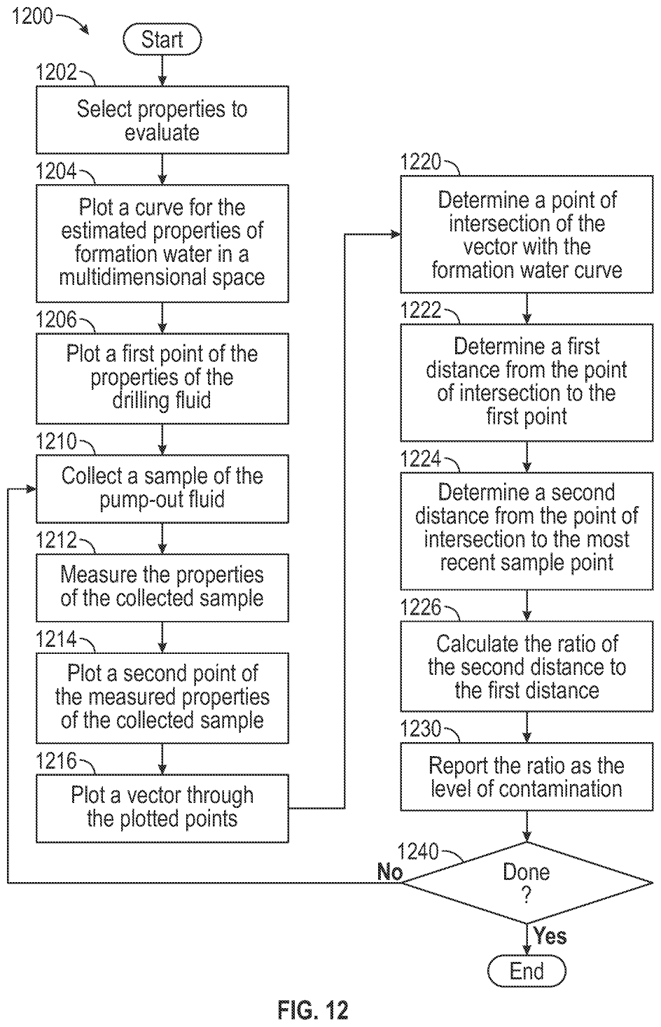

FIG. 11 is a graph illustrating a 2D example method of determining a level of contamination, in accordance with various aspects of the subject technology. While the method disclosed herein is applicable to evaluation of multiple variables in a multidimensional space, the example of FIG. 11 considered only two variables and is plotted on a two dimensional (2D) space for simplicity and clarity of the explanation.

Formation water in siliciclastic formations, which are formed in the fragments of older rocks such as sandstones and shales, often resembles concentrated seawater. This is largely due to the geological history of these formations. Many siliciclastic formations were deposited in marine environments, where seawater was trapped in the pore spaces of the sediment. Over time, this seawater evolved into formation water, retaining much of its original composition but becoming more concentrated due to processes such as chemical evaporation, diagenesis, and water-rock interactions. As a result, the major components of formation water in siliciclastic formations are typically the same as those in seawater, namely sodium and chloride ions, with smaller amounts of other ions such as calcium, magnesium, potassium, sulfate, and bicarbonate. The absolute concentrations of these ions can vary depending on factors such as the age of the formation, the depth and temperature of the reservoir, and the history of fluid migration. However, the relative proportions of the ions tend to remain fairly constant, reflecting their proportions in seawater. This characteristic composition of formation water in siliciclastic formations provides a useful baseline for contamination estimation during formation testing.

FIG. 11 depicts a example of using the disclosed deterministic method of determining a level of contamination formations for a wellbore in a siliciclastic formation. Two key properties of the fluid are evaluated, in this example density and chloride concentration. This 2D example may be conveniently visualized on a graph 1100 in which density is plotted against the vertical axis and chloride concentration is plotted against the horizontal axis. In certain embodiments, the method is implemented numerically using known mathematical techniques to define, manipulate, and evaluate multidimensional data.

In this disclosure, the term “curve” means a multidimensional surface in a multidimensional space. In a 2D space, a curve is a 2D line that may be straight or not-straight with one or more portions that are convex or concave and variable local radii of curvature. In a 3D space, a curve is a 3D surface that may be a flat plane or an undulating surface with local peaks and valleys. In higher-order spaces, a curve is not easy to visualize but still defines a feature having the appropriate number of dimensions.

The properties of the pure drilling fluid filtrate are known and are plotted as point 1120 on graph 1100. In certain embodiments, the properties of the drilling fluid vary during the course of the pump-out and would be plotted as a line instead of a point. The properties of the formation water are not known exactly but can be estimated based on the assumption that the formation water in siliciclastic formations is similar to concentrated seawater, which is primarily composed of sodium chloride. This assumption allows us to define a curve 1110 on graph 1100 that represents the expected properties of the formation water. In certain embodiments, proxies from the field are used to fine tune the sodium chloride assumption.

During the pump-out process, the properties of the fluid being sampled are measured in real-time using sensors on the formation tester. These measurements are plotted as points 1122, 1124, 1126 on the graph. At the start of the pump-out, the first measurement, plotted as point 1122, is expected to be close to the drilling fluid filtrate point 1120, indicating high contamination. As the pump-out continues, the plotted measurements should move towards the formation water curve 1110, indicating decreasing contamination. In this example, the measured properties of the second sample are plotted as point 1124. The measured properties of the most recent sample are plotted as point 1126. A transition vector 1130 is plotted through the plurality of measurement points 1120, 1122, 1124, 1126. In certain embodiments, the vector is required to pass through point 1120 as the properties of the drilling fluid are known. In certain embodiments, the vector is a best-fit to a portion of the measurements of the samples. In certain embodiments, the 2D vector is a straight line. In certain embodiments, the vector is a curve.

In certain embodiments, the slope of this vector represents the rate of change of the contamination level, and the direction of the vector indicates the trend of the contamination process. In certain embodiments, the vector is modified based on known thermodynamic chemical mixing rules in order to account for nonlinear mixing.

The ultimate goal of the 2D implementation of the disclosed method is to estimate the properties of the pure formation water and the level of contamination. This is achieved by extrapolating the transition vector 1130 through the formation water line 1110. The point of intersection 1132 represents the estimated properties of the pure formation water. The distance 1134 from intersection point 1132 to the current point 1126 represents the level of contamination. In certain embodiments, this distance is measured along the vector 1130. In certain embodiments, the scale along vector 1130 is not constant. In certain embodiments, this distance is measured perpendicular to the curve 1110. By tracking this distance 1134 over time, one can monitor the progress of the pump-out and adjust the sampling process as needed to minimize contamination. This deterministic approach provides a clear, mechanistic understanding of the contamination process and allows for more accurate and reliable estimates of formation water properties and contamination levels.

The 2D method for estimating contamination in formation water samples can be extended to three or more dimensions to account for more complex scenarios, e.g., a siliciclastic formations in contact with evaporites, wherein the formation water can be a mixture of sodium chloride and another salt, e.g., potassium chloride from a sylvite evaporite. This adds an additional variable to the analysis, which can be represented by a third axis on the graph. In an example of a 3D implementation of the disclosed method, a 3D graph would have density on the vertical axis, chloride concentration on the horizontal axis, and potassium concentration on the depth axis. This creates a 3D space in which the expected properties of the formation water are plotted as a surface defined by the possible combinations of sodium chloride and potassium chloride that could be present in the formation water. The exact shape of the surface will depend on the specific composition of the evaporite and the geochemical conditions in the reservoir. However, it can generally be approximated as a plane, or a slightly curved surface to account for molar volume mixing effects. The properties of the drilling fluid filtrate is plotted as a point in the 3D space analogous to the 2D example of FIG. 11 and the measured properties of the fluid samples are also plotted as points

This method largely works because the evaporate acts as another “pure” endmember in a mixing problem. The composition of the endmember will largely be known before the formation tester pump-out either by a priory field knowledge or mineralogical or elemental logging such as nuclear spectroscopy or surface data logging. For an application having three unknowns, e.g., the mixing ratio of contamination, the initial formation fluid, and the evaporite solvation, at least three linearly independent (rank sufficient) measurements must be made.

The method can further be extended into more than 3 dimensions. Carbonate reservoirs are more complex in that the variation of carbonate reservoir composition is large over a given region. This requires a refinement to enable a similar procedure in order to determine water based filtrate contamination in a water based formation fluid. The method for estimating contamination in formation water samples from carbonate formations builds upon the 2D and 3D methods and introduces a new element to account for the unique characteristics of carbonates. Unlike siliciclastic formations, which are primarily composed of sodium chloride, carbonate formations can have a more varied composition due to the influence of factors such as the type of carbonate mineral, the degree of dolomitization, and the geochemical conditions in the reservoir. This makes it more challenging to define a reference line or surface for the expected properties of the formation water. However, this challenge can be addressed through the use of statistical analysis and local data.

In this method, local data from other formation fluids in the same field or basin is used to define a reference plane (or slightly curved surface) in the three-dimensional space of density, chloride concentration, and another ion concentration, such as calcium. This surface represents the expected properties of the formation water based on the local geology and geochemistry. The exact shape of the surface is determined through statistical analysis, such as principal component analysis, which identifies the most significant variations in the formation water properties and uses these to define the surface. This approach takes into account the complex interactions between different factors, such as temperature, pressure, and formation composition, and provides a more nuanced understanding of the formation water

The 2D, 3D, and carbonate formation methods for estimating contamination in formation water samples are all extensions of the same fundamental approach. At its core, the method involves the analysis of the properties of the fluid being sampled in a multidimensional space and tracking the transition from the drilling fluid filtrate composition towards the formation water composition. In the 2D example of FIG. 11, the formation water line is based on the assumption that the formation water in siliciclastic formations is similar to concentrated seawater. In the 3D example, the formation water surface is defined by the possible combinations of sodium chloride and another salt, such as potassium chloride, in formations in contact with evaporites. In the carbonate formation example, the surface is defined using local data and statistical analysis to account for the more varied composition of carbonate formations. Despite these differences, all three methods share the same underlying principle of using real-time measurements to track the contamination process and estimate the properties of the pure formation water. This unifying principle provides a robust and flexible framework for contamination estimation in a wide range of formation types and conditions. The methods discussed herein are extendable to any number of formation types in any number of fields and combinations of formations therein.

In summary, the disclosed systems and methods provide a deterministic method of using measurements of multiple properties of samples of pump-out fluid to estimate one or more properties of the pure formation fluid and determining a level of contamination of the formation fluid with a second fluid, e.g., drilling fluid.

For clarity of explanation, in some instances the present technology may be presented as including individual functional blocks including functional blocks comprising devices, device components, steps or routines in a method embodied in software, or combinations of hardware and software.

In the foregoing description, aspects of the application are described with reference to specific embodiments thereof, but those skilled in the art will recognize that the application is not limited thereto. Thus, while illustrative embodiments of the application have been described in detail herein, it is to be understood that the disclosed concepts may be otherwise variously embodied and employed, and that the appended claims are intended to be construed to include such variations, except as limited by the prior art. Various features and aspects of the above-described subject matter may be used individually or jointly. Further, embodiments can be utilized in any number of environments and applications beyond those described herein without departing from the broader spirit and scope of the specification. The specification and drawings are, accordingly, to be regarded as illustrative rather than restrictive. For the purposes of illustration, methods were described in a particular order. It should be appreciated that in alternate embodiments, the methods may be performed in a different order than that described.

Where components are described as being “configured to” perform certain operations, such configuration can be accomplished, for example, by designing electronic circuits or other hardware to perform the operation, by programming programmable electronic circuits (e.g., microprocessors, or other suitable electronic circuits) to perform the operation, or any combination thereof.

The various illustrative logical blocks, modules, circuits, and algorithm steps described in connection with the examples disclosed herein may be implemented as electronic hardware, computer software, firmware, or combinations thereof. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present application.

The techniques described herein may also be implemented in electronic hardware, computer software, firmware, or any combination thereof. Such techniques may be implemented in any of a variety of devices such as general purposes computers, wireless communication device handsets, or integrated circuit devices having multiple uses including application in wireless communication device handsets and other devices. Any features described as modules or components may be implemented together in an integrated logic device or separately as discrete but interoperable logic devices. If implemented in software, the techniques may be realized at least in part by a non-volatile computer-readable memory, or other data storage medium, comprising program code including instructions that, when executed, performs one or more of the method, algorithms, and/or operations described above. The computer-readable data storage medium may form part of a computer program product, which may include packaging materials.

A computer-readable memory, as used herein, includes any type of storage media, e.g., a random access memory (RAM), a synchronous dynamic random access memory (SDRAM), a read-only memory (ROM), a non-volatile random access memory (NVRAM), an electrically erasable programmable read-only memory (EEPROM), a FLASH memory, magnetic or optical data storage media, and the like. The techniques additionally, or alternatively, may be realized at least in part by a computer-readable communication medium that carries or communicates program code in the form of instructions or data structures and that can be accessed, read, and/or executed by a computer, such as propagated signals or waves.

Other embodiments of the disclosure may be practiced in network computing environments with many types of computer system configurations, including personal computers, hand-held devices, multi-processor systems, microprocessor-based or programmable consumer electronics, network PCs, minicomputers, mainframe computers, and the like. Embodiments may also be practiced in distributed computing environments where tasks are performed by local and remote processing devices that are linked (either by hardwired links, wireless links, or by a combination thereof) through a communications network. In a distributed computing environment, program modules may be located in both local and remote memory storage devices.

In the above description, terms such as “upper,” “upward,” “lower,” “downward,” “above,” “below,” “downhole,” “uphole,” “longitudinal,” “lateral,” and the like, as used herein, shall mean in relation to the bottom or furthest extent of the surrounding wellbore even though the wellbore or portions of it may be deviated or horizontal. Correspondingly, the transverse, axial, lateral, longitudinal, radial, etc., orientations shall mean orientations relative to the orientation of the wellbore or tool. Additionally, the illustrate embodiments are illustrated such that the orientation is such that the right-hand side is downhole compared to the left-hand side.

The term “coupled” is defined as connected, whether directly or indirectly through intervening components, and is not necessarily limited to physical connections. The connection can be such that the objects are permanently connected or releasably connected. The term “outside” refers to a region that is beyond the outermost confines of a physical object. The term “inside” indicates that at least a portion of a region is partially contained within a boundary formed by the object. The term “substantially” is defined to be essentially conforming to the particular dimension, shape or another word that substantially modifies, such that the component need not be exact. For example, substantially cylindrical means that the object resembles a cylinder, but can have one or more deviations from a true cylinder.

The phrase “urging an object” or similar means the application of a force to the object in a manner that will try and move the object toward a defined position or in a specific direction without implying that the object moves or that the object is restricted from moving in another direction, even backward with respect to the direction of the applied force.

Claim language reciting “an item” or similar language indicates and includes one or more than one of the items. For example, claim language reciting “a part” means one part or multiple parts.

Moreover, claim language reciting “at least one of” a set indicates that one member of the set or multiple members of the set satisfy the claim. For example, claim language reciting “at least one of A and B” means A, B, or A and B.

Although a variety of information was used to explain aspects within the scope of the appended claims, no limitation of the claims should be implied based on particular features or arrangements, as one of ordinary skill would be able to derive a wide variety of implementations. Further and although some subject matter may have been described in language specific to structural features and/or method steps, it is to be understood that the subject matter defined in the appended claims is not necessarily limited to these described features or acts. Such functionality can be distributed differently or performed in components other than those identified herein. The described features and steps are disclosed as possible components of systems and methods within the scope of the appended claims.

Statements of the disclosure include:

(A1) A method of determining a level of contamination of a formation fluid by a drilling fluid, comprising: defining a first curve of a range of values for a plurality of properties of the formation fluid in a multidimensional space, wherein each dimension is associated with one of the plurality of properties; defining a first point of the values of the plurality of properties for the drilling fluid in the multidimensional space; accessing measurements of the plurality of properties for a plurality of samples of a fluid being pumped out of a wellbore; defining a plurality of second points of the respective measured properties in the multidimensional space; defining a vector from the first point through the plurality of second points to the first curve; determining a first distance from the first point along the vector to the first curve; determining a second distance from the most recent second point to the first curve; calculating a ratio of the second distance to the first distance; and providing the ratio as the level of contamination of the formation fluid by the drilling fluid.

(A2) The method of A1, wherein the step of defining the vector comprises determining a best-fit of a linear vector to the plurality of second points.

(A3) The method of A2, wherein the step of defining the vector requires that the vector pass through the first point.

(A4) The method of A3, wherein the second distance is measured parallel to the first distance.

(A5) The method of A1, further comprising: determining a point of intersection of the vector with the first curve; and reporting the values of the parameters at the point of intersection as the composition of the formation fluid.

(A6) The method of A1, wherein the plurality of properties of the formation fluid are based on the type of formation.

(A7) The method of A6, wherein the plurality of properties of the formation fluid are that of concentrated sea water and the type of formation is siliciclastic.

(A8) The method of A1, further comprising: collecting the plurality of samples of a fluid being pumped out of a wellbore; measuring the plurality of properties for each of the plurality of samples;

(B9) A system for determining a level of contamination of a formation fluid by a drilling fluid, comprising: a body configured to be disposed in a wellbore; a fluid analysis module coupled to the body and configured to provide measurements of at least two properties of a fluid in the wellbore; a processor coupled to the fluid analysis module and configured to receive the measurements of the at least two properties; and a memory coupled to the processor and comprising instructions that, when loaded into the processor and executed, cause the processor to execute steps: defining a first curve of a range of values for a plurality of properties of the formation fluid in a multidimensional space, wherein each dimension is associated with one of the plurality of properties; defining a first point of the values of the plurality of properties for the drilling fluid in the multidimensional space; accessing measurements of the plurality of properties for a plurality of samples of a fluid being pumped out of a wellbore; defining a plurality of second points of the respective measured properties in the multidimensional space; defining a vector from the first point through the plurality of second points to the first curve; determining a first distance from the first point along the vector to the first curve; determining a second distance from the most recent second point to the first curve; calculating a ratio of the second distance to the first distance; and providing the ratio as the level of contamination of the formation fluid by the drilling fluid.

(B10) The memory of claim 9, wherein the step of defining the vector comprises determining a best-fit of a linear vector to the plurality of second points.

(B11) The memory of claim 10, wherein the step of defining the vector requires that the vector pass through the first point.

(B12) The memory of claim 11, wherein the second distance is measured parallel to the first distance.

(B13) The memory of claim 9, further comprising: determining a point of intersection of the vector with the first curve; and reporting the values of the parameters at the point of intersection as the composition of the formation fluid.

(B14) The memory of claim 9, wherein the plurality of properties of the formation fluid are based on the type of formation.

(B15) The memory of claim 14, wherein the plurality of properties of the formation fluid are that of concentrated sea water and the type of formation is siliciclastic.

(B16) The memory of claim 9, further comprising: collecting the plurality of samples of a fluid being pumped out of a wellbore; measuring the plurality of properties for each of the plurality of samples;

(C15) A memory comprising instructions that, when loaded into a processor and executed, cause the processor to execute steps: defining a first curve of a range of values for a plurality of properties of the formation fluid in a multidimensional space, wherein each dimension is associated with one of the plurality of properties; defining a first point of the values of the plurality of properties for the drilling fluid in the multidimensional space; accessing measurements of the plurality of properties for a plurality of samples of a fluid being pumped out of a wellbore; defining a plurality of second points of the respective measured properties in the multidimensional space; defining a vector from the first point through the plurality of second points to the first curve; and determining a first distance from the first point along the vector to the first curve; determining a second distance from the most recent second point to the first curve; calculating a ratio of the second distance to the first distance; and providing the ratio as the level of contamination of the formation fluid by the drilling fluid.

(C16) The system of claim 15, wherein the step of defining the vector comprises determining a best-fit of a linear vector to the plurality of second points.

(C17) The system of claim 16, wherein the step of defining the vector requires that the vector pass through the first point.

(C18) The system of claim 17, wherein the second distance is measured parallel to the first distance.

(C19) The system of claim 15, further comprising: determining a point of intersection of the vector with the first curve; and reporting the values of the parameters at the point of intersection as the composition of the formation fluid.

(C20) The system of claim 15, wherein the plurality of properties of the formation fluid are based on the type of formation.

What is claimed is:

1. A method of determining a level of contamination of a formation fluid by a drilling fluid, comprising:
defining a first curve of a range of values for a plurality of properties of the formation fluid in a multidimensional space, wherein each dimension is associated with one of the plurality of properties;
defining a first point of the values of the plurality of properties for the drilling fluid in the multidimensional space;
accessing measurements of the plurality of properties for a plurality of samples of a fluid being pumped out of a wellbore;
defining a plurality of second points of the respective measured properties in the multidimensional space;
defining a vector from the first point through the plurality of second points to the first curve;
determining a first distance from the first point along the vector to the first curve;
determining a second distance from the most recent second point to the first curve;
calculating a ratio of the second distance to the first distance; and
providing the ratio as the level of contamination of the formation fluid by the drilling fluid.

2. The method of claim 1, wherein the step of defining the vector comprises determining a best-fit of a linear vector to the plurality of second points.

3. The method of claim 2, wherein the step of defining the vector requires that the vector pass through the first point.

4. The method of claim 3, wherein the second distance is measured parallel to the first distance.

5. The method of claim 1, further comprising:
determining a point of intersection of the vector with the first curve; and
reporting the values of the parameters at the point of intersection as the composition of the formation fluid.

6. The method of claim 1, wherein the plurality of properties of the formation fluid are based on the type of formation.

7. The method of claim 6, wherein the plurality of properties of the formation fluid are that of concentrated sea water and the type of formation is siliciclastic.

8. The method of claim 1, further comprising:
collecting the plurality of samples of a fluid being pumped out of a wellbore;
measuring the plurality of properties for each of the plurality of samples;

9. A system for determining a level of contamination of a formation fluid by a drilling fluid, comprising:
a body configured to be disposed in a wellbore;
a fluid analysis module coupled to the body and configured to provide measurements of at least two properties of a fluid in the wellbore;
a processor coupled to the fluid analysis module and configured to receive the measurements of the at least two properties; and
a memory coupled to the processor and comprising instructions that, when loaded into the processor and executed, cause the processor to execute steps:
defining a first curve of a range of values for a plurality of properties of the formation fluid in a multidimensional space, wherein each dimension is associated with one of the plurality of properties;
defining a first point of the values of the plurality of properties for the drilling fluid in the multidimensional space;
accessing measurements of the plurality of properties for a plurality of samples of a fluid being pumped out of a wellbore;
defining a plurality of second points of the respective measured properties in the multidimensional space;
defining a vector from the first point through the plurality of second points to the first curve;
determining a first distance from the first point along the vector to the first curve;
determining a second distance from the most recent second point to the first curve;
calculating a ratio of the second distance to the first distance; and
providing the ratio as the level of contamination of the formation fluid by the drilling fluid.

10. The memory of claim 9, wherein the step of defining the vector comprises determining a best-fit of a linear vector to the plurality of second points.

11. The memory of claim 10, wherein the step of defining the vector requires that the vector pass through the first point.

12. The memory of claim 11, wherein the second distance is measured parallel to the first distance.

13. The memory of claim 9, further comprising:
determining a point of intersection of the vector with the first curve; and
reporting the values of the parameters at the point of intersection as the composition of the formation fluid.

14. The memory of claim 9, wherein the plurality of properties of the formation fluid are based on the type of formation.

15. The memory of claim 14, wherein the plurality of properties of the formation fluid are that of concentrated sea water and the type of formation is siliciclastic.

16. The memory of claim 9, further comprising:
collecting the plurality of samples of a fluid being pumped out of a wellbore;
measuring the plurality of properties for each of the plurality of samples;

15. A memory comprising instructions that, when loaded into a processor and executed, cause the processor to execute steps:
defining a first curve of a range of values for a plurality of properties of the formation fluid in a multidimensional space, wherein each dimension is associated with one of the plurality of properties;
defining a first point of the values of the plurality of properties for the drilling fluid in the multidimensional space;
accessing measurements of the plurality of properties for a plurality of samples of a fluid being pumped out of a wellbore;
defining a plurality of second points of the respective measured properties in the multidimensional space;
defining a vector from the first point through the plurality of second points to the first curve;
determining a first distance from the first point along the vector to the first curve;
determining a second distance from the most recent second point to the first curve;
calculating a ratio of the second distance to the first distance; and
providing the ratio as the level of contamination of the formation fluid by the drilling fluid.

16. The system of claim 15, wherein the step of defining the vector comprises determining a best-fit of a linear vector to the plurality of second points.

17. The system of claim 16, wherein the step of defining the vector requires that the vector pass through the first point.

18. The system of claim 17, wherein the second distance is measured parallel to the first distance.

19. The system of claim 15, further comprising:
determining a point of intersection of the vector with the first curve; and
reporting the values of the parameters at the point of intersection as the composition of the formation fluid.

20. The system of claim 15, wherein the plurality of properties of the formation fluid are based on the type of formation.
